(19) United States
(12) Patent Application Publication
(10) Pub. No.: US 2026/0235004 A1
(43) Pub. Date: Aug. 13, 2026

(54) FIELD INJECTION PORT

(71) Applicant: Velan Inc., Montreal (CA)

(72) Inventors: Vahe NAJARIAN, Montreal (CA); Edward HADJUR, Montreal (CA); Douglas Gene LAY, Chandler, AZ (US); Debajyoti MAULIK, Montreal (CA); Pierre J.A. LEGAULT, Montreal (CA); John VERNERS, Montreal (CA)

(73) Assignee: Velan Inc., Montreal (CA)

(21) Appl. No.: 19/536,710

(22) Filed: Feb. 11, 2026

Related U.S. Application Data
(60) Provisional application No. 63/757,501, filed on Feb. 12, 2025.

(51) Int. Cl. E21B 21/10; A61M 39/02; E21B 21/08; E21B 34/02

(52) U.S. Cl. CPC E21B 21/103; E21B 21/08; E21B 34/02; A61M 2039/0223

(57) ABSTRACT

A valve assembly (100) is provided with a body (102) and a field injection port (200) formed with the body (102) during manufacturing of the body (102). The field injection port (200) comprises an orientation that guides a drilling implement placed therein along a predetermined path (202) to intersect a targeted location (204).

### CROSS-REFERENCE TO RELATED APPLICATION

This application claims the benefit of U.S. Provisional Application No. 63/757,501, filed Feb. 12, 2025.

### TECHNICAL FIELD

The embodiments described below relate to the field of industrial valves and particularly to sealing valves in order to avoid primary sealing failures.

### BACKGROUND

High-pressure rotary valves, such as plug and ball valves, typically include a valve body defining a fluid passage and a rotary closure member with a through-bore mounted to selectively block or permit fluid flow. A stem rotates the closure member between an open position, where the bore aligns with the body passage, and a closed position, where the bore is oriented transversely to the passage. The stem extends from the valve interior through a stem passage that is normal to the fluid passage. In common designs, a bonnet is bolted to the valve body and cooperates with a shoulder at the bottom of the stem to prevent blowout under high internal pressure; alternatively, the stem may be inserted through one end of a single-piece body and retained by an internal abutment surface in the stem passage that engages the stem shoulder. U.S. Pat. No. 6,095,493 provides an example of a valve, describing its construction and use, and is herein incorporated by reference.

Leak Detection and Repair (LDAR) programs require emissions monitoring of pipelines, valves, fittings, flanges, and related components, with packing and bonnet/body interfaces being frequent leak points due to packing and/or gasket wear. Nonconforming valves may be repaired several times before major repair or replacement is required, and even replacing a relatively inexpensive valve can be costly due to labor and process downtime. Consequently, reusable injectable packing is increasingly used to mitigate leakage while minimizing process outages.

A service process and related products have been developed to drill into various valve portions and inject a sealing compound that stops leakage while preserving valve functionality. This process modifies existing valves, generally during field operation, by drilling into the body and/or bonnet near the packing and gasket interfaces, which introduces alignment risk that can cause unintended media release and potential injury if not executed precisely. Incorrect or inaccurate depth, angle, location, or tool sizing during such operations can render an otherwise serviceable valve unusable. Thread damage and insufficient thread engagement is also a common occurrence. Any procedural error can make the final drilling ineffective, destructive, or hazardous.

Conventional valve bodies and bonnets are designed to contain internal media and prevent atmospheric leakage, but they do not incorporate features that reduce the risks associated with injectable packing processes or facilitate optimal access for injection. There is a need for valve bodies and/or bonnets with integrated provisions that ensure proper injection location, alignment, and thread engagement while meeting applicable code requirements and reducing risk during and after injection operations.

### SUMMARY

A method of manufacturing a valve assembly is provided. The method includes forming a body, defining a field injection port in the body, configuring the field injection port to accept a drilling implement, and orienting the field injection port to guide the drilling implement along a predetermined path that intersects a targeted location in the valve assembly.

A valve assembly is provided. The valve assembly includes a body and a field injection port formed with the body during manufacturing of the body, wherein the field injection port has an orientation that guides a drilling implement placed in the field injection port along a predetermined path to intersect a targeted location.

A method of repairing a valve assembly having a body is provided. The method includes drilling an injection bore in the body through a field injection port with a drilling implement, where the field injection port is defined by the body during manufacturing of the valve assembly, guiding the drilling implement with the field injection port along a predetermined path in the body to form a drilled injection bore that intersects a targeted location, fluidically coupling the field injection port with the targeted location with the drilled injection bore, and injecting a repair fluid into the field injection port and subsequently into the targeted location via the drilled injection bore.

### ASPECTS

According to an aspect, a method of manufacturing a valve assembly comprises forming a body, defining a field injection port in the body, configuring the field injection port to accept a drilling implement, and orienting the field injection port to guide the drilling implement along a predetermined path that intersects a targeted location in the valve assembly.

Preferably, the method further comprises assembling the valve assembly using the body.

Preferably, the method comprises the body comprising a bonnet and further comprises defining the field injection port in the bonnet.

Preferably, the method comprises the body comprising a valve body and further comprises defining the field injection port in the valve body.

Preferably, the method comprises the body comprising a flange in the body and further comprises defining the field injection port in the flange.

Preferably, the method comprises the targeted location comprising a packing.

Preferably, the method comprises the targeted location comprising a valve seat.

Preferably, the method comprises the targeted location comprising a gasket.

Preferably, the method comprises the gasket comprising a body bonnet gasket.

Preferably, the method comprises the gasket comprising a body flange end gasket.

Preferably, the method comprises the gasket comprising a pressure seal gasket.

Preferably, the method comprises defining the field injection port in the body further comprising at least partially defining the field injection port using at least one of casting, forging, drilling, threading, and machining.

Preferably, the method comprises defining the field injection port in the body further comprising defining a guide chamber configured to further guide the drilling implement.

Preferably, the method comprises defining the field injection port in the body by at least partially forming the body with an additive manufacturing process.

Preferably, the method comprises defining a chip chamber between an aperture of the field injection port and a terminus of the field injection port.

Preferably, the method further comprises providing a wall defined by the body, the wall disposed between a terminus of the field injection port and the targeted location, wherein the wall comprises a material span corresponding to a predetermined wall thickness between the terminus and the targeted location.

Preferably, the method comprises the material span comprising a minimum wall thickness that meets or exceeds standards provided by at least one of ASME, API, ISO, and MSS.

Preferably, the method comprises threading the field injection port to define threads in the field injection port.

Preferably, the method comprises removably installing a plug into the field injection port and protecting the threads from damage with the plug.

According to an aspect, a valve assembly comprises a body and a field injection port formed with the body during manufacturing of the body, wherein the field injection port comprises an orientation that guides a drilling implement placed therein along a predetermined path to intersect a targeted location.

Preferably, the valve assembly comprises the body comprising a valve body that further comprises the field injection port.

Preferably, the valve assembly comprises a guide chamber of the field injection port configured to further guide the drilling implement.

Preferably, the valve assembly comprises the body comprising a body flange that further comprises the field injection port.

Preferably, the valve assembly comprises the body comprising a bonnet that further comprises the field injection port.

Preferably, the valve assembly comprises the targeted location comprising a packing.

Preferably, the valve assembly comprises the targeted location comprising a valve seat.

Preferably, the valve assembly comprises the targeted location comprising a gasket.

Preferably, the valve assembly comprises the gasket comprising a body bonnet gasket.

Preferably, the valve assembly comprises the gasket comprising a body flange end gasket.

Preferably, the valve assembly comprises the gasket comprising a pressure seal gasket.

Preferably, the valve assembly comprises the field injection port being at least partially formed in the body using at least one of casting, forging, drilling, threading, and machining.

Preferably, the valve assembly comprises the field injection port being at least partially formed in the body with an additive manufacturing process.

Preferably, the valve assembly comprises a chip chamber defined between an aperture of the field injection port and a terminus of the field injection port, wherein the chip chamber comprises a space having a cross-sectional area greater than a cross-sectional area of the drilling implement, and wherein the chip chamber is configured to capture chips created during a drilling operation.

Preferably, the valve assembly comprises threads defined in the field injection port.

Preferably, the valve assembly comprises at least one of a gate valve, a globe valve, a check valve, a ball valve, a butterfly valve, and a plug valve.

Preferably, the valve assembly comprises a wall defined by the body disposed between a terminus of the field injection port and the targeted location, wherein the wall comprises a material span corresponding to a predetermined wall thickness between the terminus and the targeted location.

Preferably, the valve assembly comprises the material span comprising a minimum wall thickness that meets or exceeds standards provided by at least one of ASME, API, ISO, and MSS.

According to an aspect, a method of repairing a valve assembly having a body comprises drilling an injection bore in the body through a field injection port with a drilling implement, the field injection port being defined by the body during manufacturing of the valve assembly, guiding the drilling implement with the field injection port along a predetermined path in the body to form a drilled injection bore that intersects a targeted location, fluidically coupling the field injection port with the targeted location with the drilled injection bore, and injecting a repair fluid into the field injection port and subsequently into the targeted location via the drilled injection bore.

Preferably, the method comprises the body comprising a valve body that further comprises the field injection port.

Preferably, the method comprises the body comprising a body flange that further comprises the field injection port.

Preferably, the method comprises the body comprising a bonnet that further comprises the field injection port.

Preferably, the method further comprises collecting chips produced by drilling into the body, wherein the chips are collected in a chip chamber defined by the field injection port.

Preferably, the method comprises guiding the drilling implement with a guide chamber of the field injection port.

Preferably, the method further comprises removing a plug preinstalled in the field injection port.

Preferably, the method comprises the targeted location comprising a packing.

Preferably, the method comprises the targeted location comprising a valve seat.

Preferably, the method comprises the targeted location comprising a gasket.

Preferably, the method comprises the gasket comprising a body bonnet gasket.

Preferably, the method comprises the gasket comprising a body flange end gasket.

Preferably, the method comprises the gasket comprising a pressure seal gasket.

Preferably, the method comprises the targeted location comprising at least one of a packing and a gasket of the valve assembly.

Preferably, the method comprises the drilled injection bore passing through a wall defined by the body, wherein the wall comprises a material span corresponding to a predetermined wall thickness between the terminus and the targeted location.

Preferably, the method comprises the material span comprising at least a minimum wall thickness that meets or exceeds standards provided by at least one of ASME, API, ISO, and MSS.

### BRIEF DESCRIPTION OF THE DRAWINGS

FIG. 1 illustrates a valve assembly with a field injection port according to an embodiment.

FIG. 2 illustrates a partial cutaway view of a valve assembly with a field injection port according to an embodiment.

FIG. 3 illustrates a prior art valve and drill-and-tap approach.

FIG. 4 illustrates a partial cross-sectional view of a valve assembly with a field injection port according to an embodiment.

FIG. 5 illustrates a magnified view of a portion of FIG. 4.

FIG. 6 illustrates a partial cross-sectional view of a valve assembly with a field injection port according to an embodiment.

FIG. 7 illustrates a partial cross-sectional view of a valve assembly with a field injection port according to another embodiment.

FIG. 8 illustrates a magnified view of a portion of FIG. 7.

FIG. 9 illustrates a partial cross-sectional view of a valve assembly with a field injection port according to yet another embodiment.

FIG. 10 illustrates a magnified view of a portion of FIG. 9.

FIG. 11 illustrates a partial cross-sectional view of a valve assembly with a field injection port according to an additional embodiment.

FIG. 12 illustrates a magnified view of a portion of FIG. 11.

FIG. 13 illustrates a partial cross-sectional view of a valve assembly with a field injection port according to a further embodiment.

FIG. 14 is a flowchart illustrating a method of manufacturing a valve assembly according to an embodiment.

FIG. 15 is a flowchart illustrating a method of repairing a valve assembly according to an embodiment.

### DETAILED DESCRIPTION

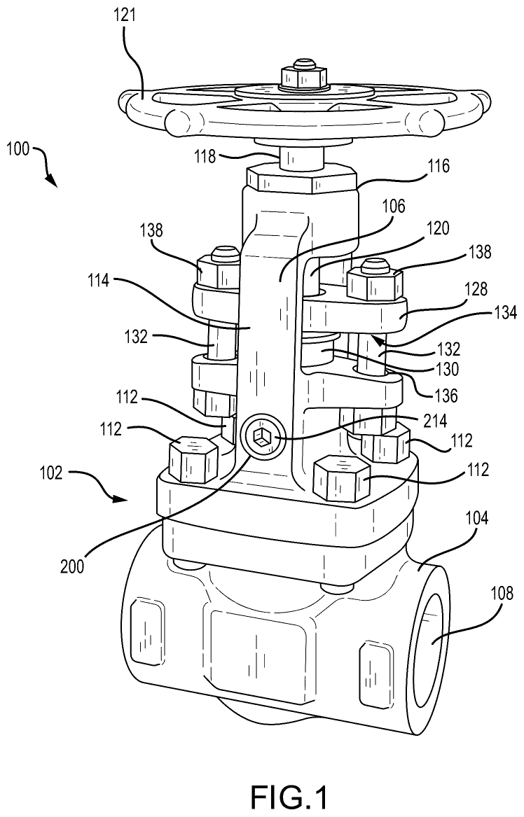

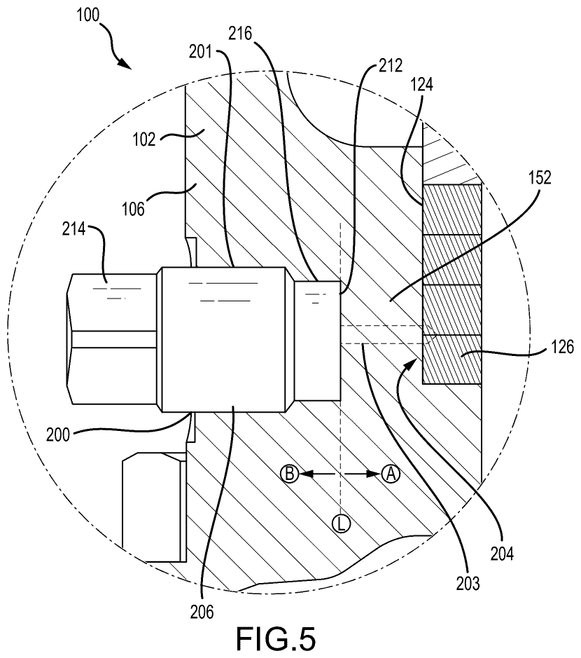

FIGS. 1, 2 and 4-15 and the following description depict specific examples to teach those skilled in the art how to make and use a valve assembly having a field injection port. For the purpose of teaching inventive principles, some conventional aspects have been simplified or omitted. Those skilled in the art will appreciate variations from these examples that fall within the scope of the present description. Those skilled in the art will appreciate that the features described below can be combined in various ways to form embodiments. Although the disclosure focuses on a valve assembly having a field injection port, it will be appreciated by those skilled in the art that the embodiments provided may be used in fields outside the valve arts. As a result, the embodiments described below are not limited to the specific examples described below, but only by the claims and their equivalents.

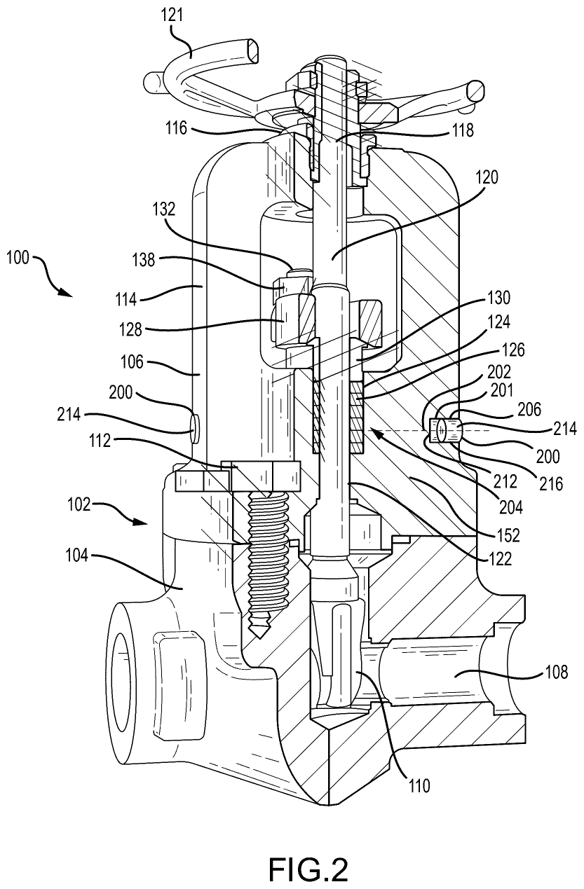

Referring to FIGS. 1 and 2, a valve assembly 100 illustrating elements of some of the present embodiments is provided. FIG. 2 illustrates the valve assembly 100 shown in cross-section to reveal internal components of the valve assembly 100 that may not be visible in FIG. 1. Some examples of valve assemblies 100 may include, but are not limited to, ASME B16.34, API, ISO, and MSS certified valves. Applications may include, but are not limited to, oil and gas, chemical, petrochemical, power, mining, process, cryogenic, LNG, water, and wastewater, nuclear, and pulp and paper, for example.

### Valve Assemblies

A gate valve is illustrated in the drawings, but other valve types are considered, such as globe, check, ball, butterfly, and plug valves, for example, and without limitation. The valve assembly 100 has a body 102 that may be a single piece or formed from multiple pieces. In some embodiments, the body 102 has a valve body 104 and a bonnet 106 attached to the valve body 104. In embodiments illustrated, the bonnet 106 is attached to the valve body with mechanical fasteners 112. The valve body 104 defines a fluid passage 108 in which a valve member 110 is mounted for selectively blocking and allowing fluid flow through the valve body 104.

The body 102 further includes lateral elongated support members 114 coupled to a distal shoulder 116 provided proximate an outer distal end 118 of a stem member 120. The lateral elongated support members 114 are disposed on opposed sides of the body 102 to form an integral support structure for supporting and guiding the stem member 120 during operation of the valve assembly 100. In some embodiments, the distal shoulder 116 prevents the stem member 120 from being ejected from a stem passage 122, in which the stem member 120 is rotatably coupled, due to fluid pressure inside of the body 102. In some embodiments, the lateral elongated support members 114 provide additional resistance to bending or misalignment of the stem member 120 under elevated internal fluid pressures or external side loads, thereby maintaining proper engagement between the stem member 120 and the valve member 110.

Turning to FIG. 2 in particular, the valve assembly 100 includes the stem member 120 extending through the stem passage 122 defined in the valve body 104, the stem member 120 being connected at a bottom end thereof to the valve member 110 to translate the valve member 110, along an axis perpendicular to the fluid passage 108, between an open position in which the fluid passage 108 is unobstructed and a closed position in which the valve member 110 blocks the fluid passage 108. An actuation member 121, such as a wheel, lever, and coupler for any device, manual or automated, that provides a force for valve translation is provided.

The valve assembly 100 further includes an annular packing chamber 124 defined between the stem member 120 and the stem passage 122. The packing chamber 124 is filled by a packing 126, such as a plurality of packing rings disposed between extrusion rings, for example and without limitation. The packing 126 is configured to prevent leakage from the fluid passage 108 about the stem member 120. The valve assembly 100 also includes a gland assembly 128 configured to maintain the packing 126 under axial compression. The gland assembly 128 may include a gland bushing 130, such as a split gland bushing, mounted on the stem member 120 and positioned over an outer surface of the packing, such as a top extrusion ring, for example. Other members, washers, springs, bushings, bearings, hardware, etc. may be present, but are omitted for clarity. For example, a plurality of Belleville washers (not shown) may be disposed about the gland bushing 130 to provide a spring force that enables self-adjustment to compensate for wear of the packing 126.

In some embodiments, the valve assembly 100 includes threaded studs 132 connecting the gland assembly 128 to the valve body 104, the threaded studs 132 extending through respective axially extending lateral bores 134 defined in a gland member of the gland assembly 128 and into corresponding bores 136 defined in a portion of the valve body 104 surrounding the stem passage 122. The valve assembly 100 also includes packing adjustment nuts 138 threadingly engaged with upper ends of the threaded studs 132, the packing adjustment nuts 138 being configured to compress the packing 126, and any Belleville washers or other elements that may be present, thereby causing the gland assembly 128 to apply a compressive load on the packing 126.

Packing 126 functions as dynamic sealing positioned between the stem member 120 and a valve bonnet cavity, minimizing leakage pathways while permitting controlled stem rotation or axial movement. The selection of packing 126 is determined by the specific operating parameters including temperature range, system pressure, and the chemical composition of the process fluid. In modern LDAR-compliant valve assemblies 100, packing 126 is frequently supplied in pre-cut, pre-measured ring sets that ensure precise fit and consistent compression characteristics, thereby meeting stringent fugitive emission standards such as API 622 specifications, for example and without limitation.

In some embodiments, packing 126 is made from flexible graphite, particularly in high-temperature and high-pressure refinery environments. Graphite-based packings 126 demonstrate thermal stability at temperatures exceeding 650° C. and exhibit chemical inertness across a broad spectrum of industrial fluids. In some embodiments, to enhance mechanical properties and longevity, graphite based packing 126 is reinforced with other materials, such as Inconel® or carbon fiber, for example. This provides composite structures that aid structural support and reduce wear characteristics under dynamic operating conditions.

In some embodiments, polytetrafluoroethylene (PTFE) serves as an alternative or complementary packing material for the packing 126, particularly in applications involving corrosive chemical environments and moderate temperature ranges. PTFE exhibits exceptional chemical resistance and provides low-friction sealing surfaces that are conducive to frequent valve operation of the valve assembly 100. In some embodiments, PTFE-based packings 126 are filled with reinforcing materials including glass fibers, carbon particulates, or bronze powder, for example, and without limitation. This improves mechanical performance and mitigates cold-flow tendencies under sustained pressure. In some embodiments, packing 126 may employ layered configurations combining graphite and PTFE rings.

In some embodiments, elastomeric sealing elements are employed as part of the packing 126, such as in moderate-temperature and moderate-pressure applications where flexibility and conformability are advantageous. These materials are formulated to provide chemical resistance to specific process fluids while maintaining the elasticity necessary for effective sealing at the stem interface between the stem member 120 and the stem passage 122. It will be appreciated that other packing 126 known in the art are contemplated, and combinations of packing 126 are considered in embodiments.

Valve body-to-bonnet gasket materials provide static sealing between stationary valve components, particularly at the body-to-bonnet interface between the valve body 104 and the bonnet 106, although gaskets disposed in other locations of the valve assembly 100 are considered. Gasket selection is determined by system pressure, operating temperature, and the corrosiveness of the contained fluid. In an embodiment, gaskets may have various sizes, dimensions, configurations, and material compositions, including flat, round, and oval profiles, for example. Non-metallic, metallic, and combinations thereof are considered in some embodiments.

Non-metallic gaskets are typically employed in low-pressure and low-temperature applications. In some embodiments, rubber-based gaskets, such as those formulated from nitrile elastomers, for example and without limitation, provide effective sealing and oil-resistant properties at the interface between the valve body 104 and the bonnet 106 or other mating components of the valve assembly 100. In some embodiments, gaskets contain compressed non-asbestos fiber (CNAF), which are generally composed of aramid and mineral fibers bound with elastomeric binders. This provides enhanced compressibility and chemical resistance. In some embodiments, graphite-based non-metallic gaskets are utilized, and provide thermal stability in applications where temperatures reach around 460° C. In some embodiments, PTFE gaskets are utilized, as they exhibit excellent chemical inertness for non-metallic applications.

Metallic gaskets, on the other hand, are used in some embodiments. Metallic gaskets are beneficial in high-pressure and high-temperature environments, which are characteristics often found in oil and gas operations or nuclear applications, for example. In some embodiments, metallic gaskets are fabricated from corrosion-resistant metals including stainless steel, low-carbon steel, soft iron, or aluminum alloys. Metallic gaskets may have oval or octagonal cross-sectional geometry, in some embodiments, as this, in addition to high-tension connections such as the mechanical fasteners 112, generates the contact pressure necessary for effective sealing.

In some embodiments, spiral-wound gaskets represent a hybrid material, combining metal wire windings with non-metallic filler materials to provide improved sealing performance under elevated pressure and temperature conditions. Ring joint gaskets, fabricated entirely from metal, may be employed in flanged connections associated with the valve body 104.

It will be appreciated that other gasket materials known in the art are contemplated, and combinations of gasket materials are considered in embodiments. Although example applications for packing 126 and gasket materials are provided, these shall not limit the embodiments to the particular applications described.

### Field Injection Port

In some embodiments, a field injection port 200 is provided. In order to repair packing 126 and gaskets in the field, injection methodologies are also provided in some embodiments. The field injection port is a small port chamber that can be made to accept a potential drilling implement. Unless drilled through during use, such as during field servicing, the chamber itself is only open to the exterior of the valve and does not connect with the internal workings of the valve, as will be described below.

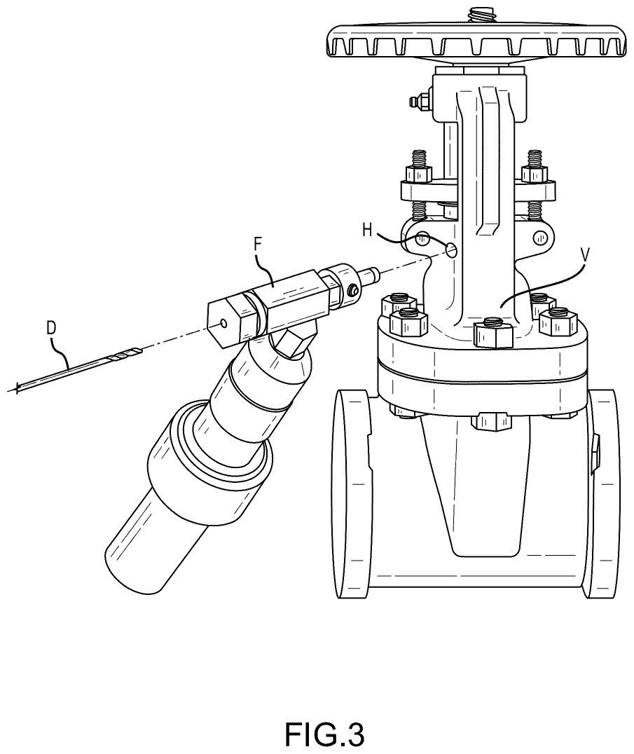

FIG. 3 depicts a prior-art valve and drill-and-tap approach for valve repair while a valve is in the field. A drill bit is used to create a hole (H) that is subsequently tapped at a location proximate to the packing or gasket. This generally occurs in the field while the valve (V) is in place where it is installed. This operation is delicate and finicky because incorrect depth, angle, location, or tool sizing can render an otherwise serviceable valve unusable. Not only does this destroy the valve, but the costs incurred for the replacement, testing, and halting of processes can be prohibitive. Drilling and tapping into curved or irregular surfaces by hand and without elaborate fixtures, which are impractical in the field, does not lend itself to accurate results. An injection “gun” or fitting (F) is also utilized, which may strip threads if engagement is inadequate or if the tapped threads are of low quality, and can be rotated easily after installation, increasing the risk of accidental unthreading or thread damage when thread engagement is insufficient. The final steps of such a procedure entail drilling through the fitting with another drill bit (D), into the valve (V), to form a passage for introducing or augmenting packing. Any procedural error during this multi-step process can make the final drilling ineffective, destructive, and/or hazardous.

Turning back to the present embodiments, injectable repair fluid compositions are administered to repair or arrest leakage while the valve assembly 100 remains in operational service, in some embodiments. These compositions vary in their formulation based on the leak severity and operational requirements. For example, minor leakage, typically resulting from small surface scratches or localized corrosion at sealing interfaces such as around the stem member 120 or between the valve body 104 and the bonnet 106, may be addressed with enhanced-sealing oil-based compositions comprising a mineral or synthetic base oil combined with thickening agents and potentially corrosion inhibitors. These formulations provide incremental sealing improvement through their viscosity and film-forming characteristics. For more substantial leakage pathways resulting from deeper surface damage or voids, heavier sealant compositions incorporating solid particulate fillers are employed in some embodiments. These compositions typically contain PTFE or other fillers and flakes distributed throughout a liquid matrix, whereby the solid fillers mechanically occlude the leak pathway. In some embodiments, reactive polymer sealants, including polyurethanes and/or acrylate resin formulations, provide alternative repair approaches. These compositions are injected into the leak site where they undergo hydrophilic reaction with ambient moisture and subsequently expand to form flexible, elastomeric seals. The reaction kinetics and foam expansion characteristics of such formulations may be modulated to accommodate specific leak geometry and environmental conditions. Hydrophilic polyurethane foams are particularly suited for persistently moist operating environments. In some embodiments, non-foaming polyurethane resin compositions may be re-injected following initial foam application to improve long-term leak arrest. It will be appreciated that other repair fluids, solutions, compositions, and mixtures, with or without added solids, alone or in combination are considered in the embodiments provided herein.

With continuing reference to FIGS. 1 and 2, the field injection port 200 is illustrated as being formed with the body 102, and specifically the bonnet 106. The field injection port 200 is a structure formed in the body 102 of the valve assembly 100 during manufacture of the body 102 to provide a predetermined, code-compliant access path into a pressure boundary for delivery of repair fluids to a targeted location 204 inside the valve assembly 100. In embodiments, the field injection port has a bore 201. In some embodiments, the field injection port has a cylindrical bore 201. In some embodiments, the field injection port has a conical and/or frustoconical bore 201. In some embodiments, the field injection port has multiple portions having combinations of cylindrical and conical and/or frustoconical bore 201. It will be understood that other cross-sectional bore 201 profiles and/or combinations of bore 201 profiles may be present in embodiments. The bore 201 may be normal to the surface on which it is defined, or non-normal.

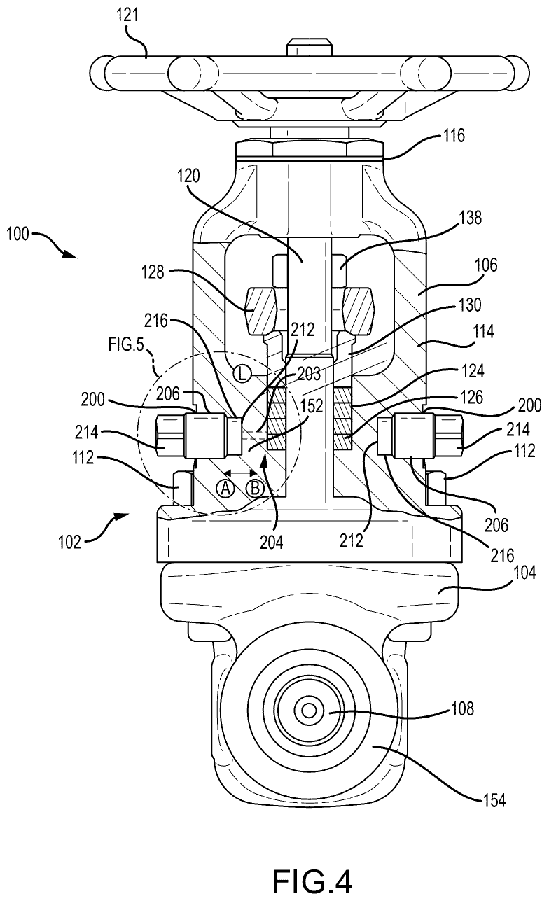

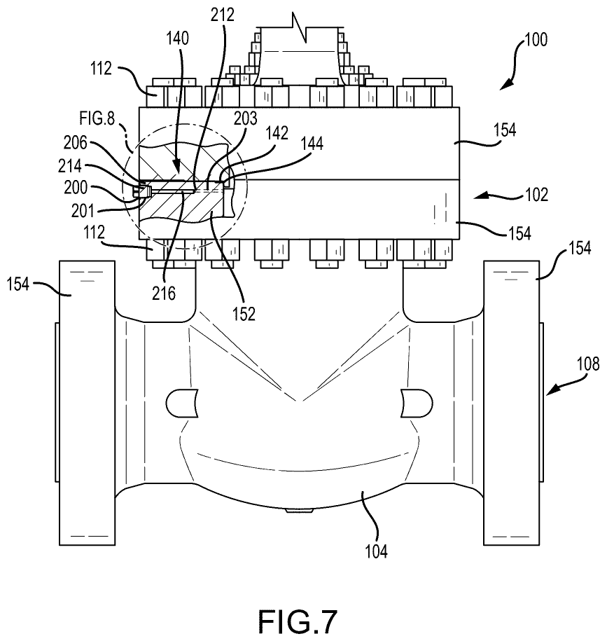

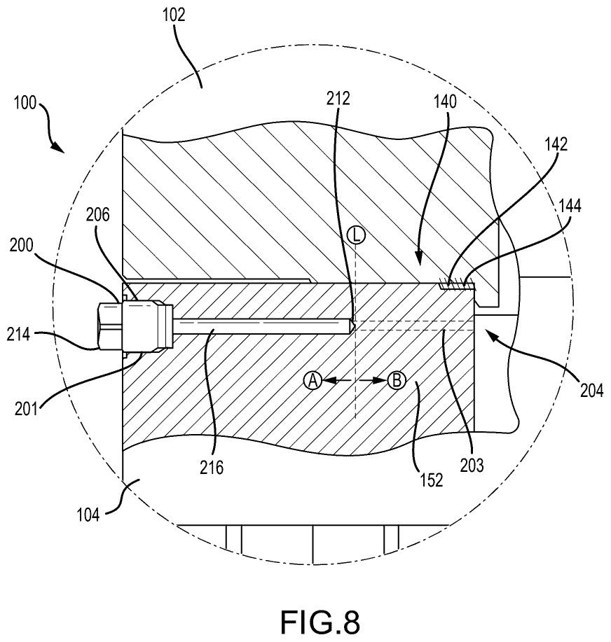

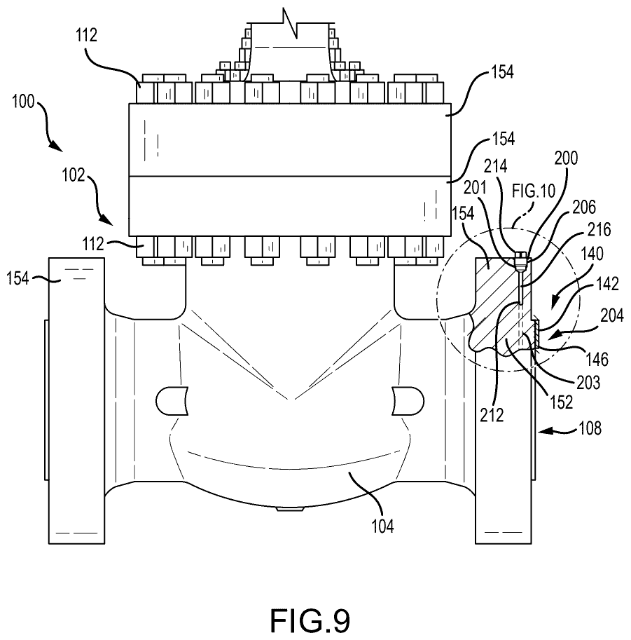

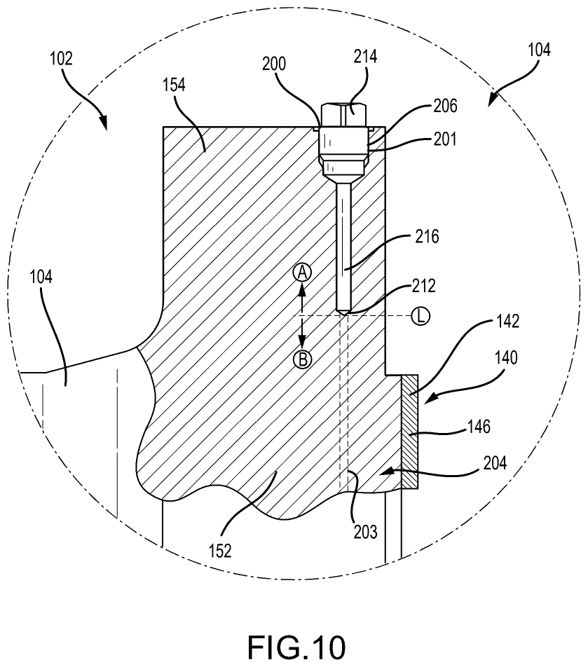

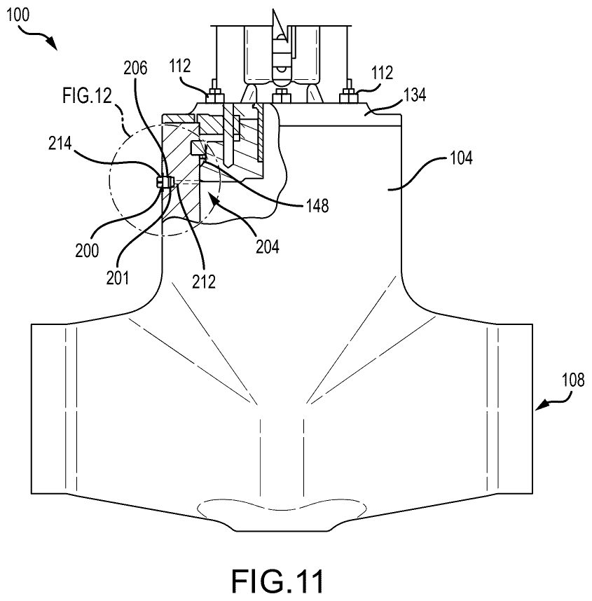

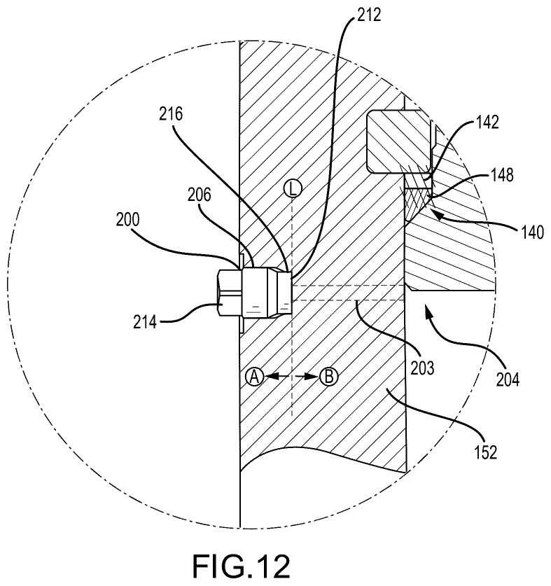

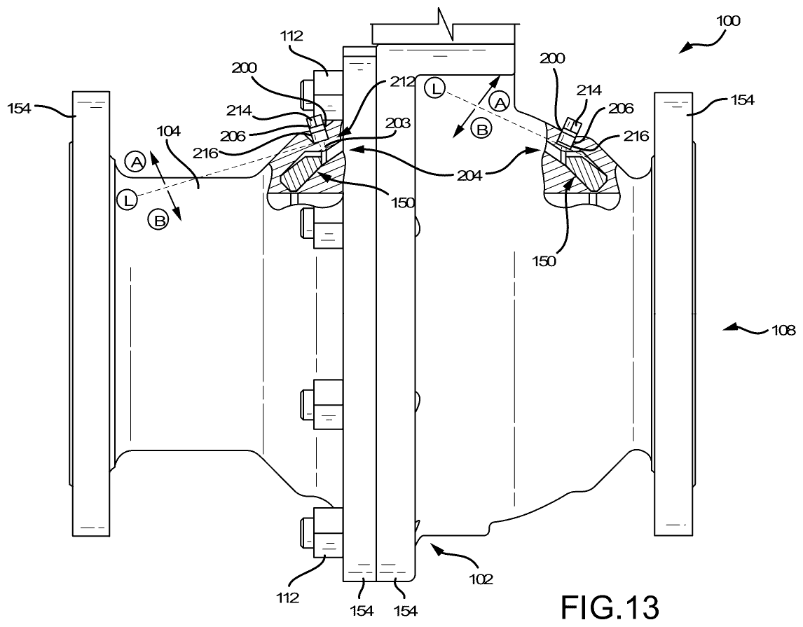

Turning additionally to FIGS. 4-13, the field injection port 200 includes an orientation that is configured to guide a drilling implement along a predetermined path 202 toward the targeted location 204, such as the packing chamber 124 containing the packing 126 (FIGS. 1, 2, 4-6). The targeted location 204 can also include a gasket interface 140 having a gasket 142, such as one or more of a body-to-bonnet gasket 144 (FIGS. 7-8), a body-to-flange-end gasket 146 (FIGS. 9-10), or a pressure seal gasket 148 (FIGS. 11-12) in some embodiments. In some embodiments, the targeted location 204 is a valve seat 150 associated with the valve member 110 (FIG. 13). Any location in the valve assembly where a repair fluid may be delivered may be considered a targeted location 204. This includes regions proximate any of the regions/features noted as a targeted location 204 herein.

In some embodiments, the field injection port 200 is threaded with internal threads 206 configured to engage a mating injection fitting, plug 214, or drilling guide member. The internal threads 206 may be configured as National Pipe Taper (NPT) threads, or as other thread forms suitable for high-pressure fittings and operations. The internal threads 206 provide the requisite thread engagement and sealing performance under operating pressure.

In some embodiments, the plug 214 removably engages the field injection port 200. The plug 214 has complimentary threads that engage the internal threads 206 of the field injection port 200. The plug 214 serves to protect the internal threads 206 from damage and prevents debris from entering the field injection port 200. The plug 214 is removed for drilling and/or fluid injection procedures. The plug 214 may be re-installed after tooling related to drilling and/or fluid injection procedures is removed from the field injection port 200.

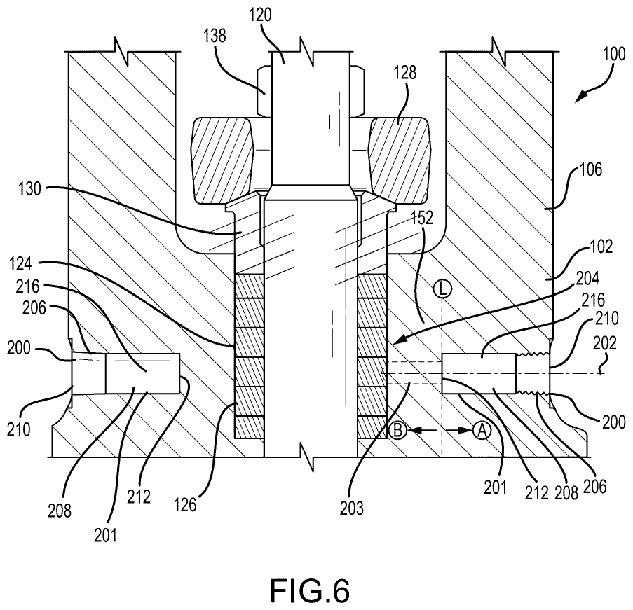

In some embodiments, the field injection port 200 includes a chip chamber 208 disposed between an aperture 210 of the bore 201 and a terminus 212 of the field injection port 200, as shown in FIG. 6. When drilling with a sealed fitting, for example, chips may not be readily ejected from the drill site. The chip chamber 208 is configured to collect/capture chips generated during a drilling operation and comprises a space configured to contain chips and swarf. In embodiments, the chip chamber 208 has a cross-sectional area greater than a cross-sectional area of the drilling implement used during the drilling operation. The increased cross-sectional area of the chip chamber 208 provides chip clearance that reduces the risk of the drill implement jamming and helps to maintains a sealed environment during drilling.

It will be appreciated that the terminus 212 of the field injection port 200 also defines a terminus of the chip chamber 208 in some embodiments. In some embodiments, a guide chamber 216 is present, being configured to guide the drilling implement to ensure drilling along the predetermined path 202 that intersects the targeted location 204, as illustrated in FIGS. 7-10. The guide chamber 216 is a pre-drilled bore that closely receives the drilling implement and constrains lateral motion so that the drilled injection bore 203 follows the predetermined path 202. The guide chamber 216 is the same diameter or smaller than the diameter of the drilling implement.

In some embodiments, both a chip chamber 208 and a guide chamber 216 are present. The guide chamber may also comprise a chamber that accepts a guide bushing for drilling operations, in some embodiments. An example of the kind of arrangement where a guide bushing may be accepted by the field injection port 200 is found in FIG. 6.

The field injection port 200 may be at least partially formed in the body 102 using casting, forging, drilling, threading, machining, or combinations thereof. In some embodiments, the field injection port 200 is at least partially formed in the body 102 using an additive manufacturing process, enabling complex internal geometries such as contoured or baffled chip chambers and drilling paths. In some embodiments, the field injection port 200 is threaded with internal threads 206, which may be configured as NPT threads or as other thread forms suitable for high-pressure injection fittings, in order to provide sufficient thread engagement and sealing performance under operating pressures. Threading may encompass, drilling, thrilling, electrical discharge machined (EDM) threading, and deformative methods, for example, in addition to other threading operations known in the art.

In embodiments, the body 102 defines a wall 152 disposed between the terminus 212 of the field injection port 200 or chip chamber 208 or guide chamber 216 and the targeted location 204. In FIGS. 2, 5, 6, 8, 10, 12, and 13, a dashed line (L) indicates a border of the terminus 212. The wall 152 comprises a material span corresponding to a predetermined wall thickness between the terminus 212 and the targeted location 204. The material span begins at the side of the line (L) denoted by arrow (B). The side of the arrow denoted by arrow (A) is the region wherein the field injection port 200 is defined. For example, a guide chamber 216 or chip chamber 208 will be on the (B) side of line (L). In some embodiments, the predetermined wall thickness is selected to meet or exceed minimum wall thickness standards for pressure-retaining components provided by established safety/engineering standards. In some embodiments, the wall thickness meets or exceeds standards provided by at least one of American Society of Mechanical Engineers (ASME), American Petroleum Institute (API), (International Organization for Standardization) ISO, (Manufacturers Standardization Society) MSS, EN (European Norm), and other standards known in the art. For example, ASME B16.34 serves as the foundational standard for valve body wall thickness requirements, and specifies a minimum valve body wall thickness values based on valve size, pressure class, and material group, diameter of pressure boundary, etc. API standards, such as API 600, define minimum body wall thickness by size and pressure class, and may establish a higher safety baseline for refinery and petroleum applications than ASME B16.34 for the same pressure ratings. Other API sections may include 603, 594, 6D, 623, 602, etc., for example and without limitation. ISO standards, such as ISO 17292 and 10434 also address body wall thickness requirement. Similarly, some MSS standards refer to body wall thickness requirements. The location, size, shape, threading size, threading engagement, orientation, depth, chip chamber 208 presence and geometry and other attributes of the field injection port may also, in some embodiments, conform to ASME, API, ISO, EN, and MSS standards, and other related standards known in the art, where applicable. For example, such a standard may include EN 12516-2.

The thickness of the wall 152 is effectively the material span of the wall that remains after forming the field injection port 200. In an embodiment, the field injection port 200 is configured to minimize the amount of material required to be drilled while maintaining pressure boundary integrity and code compliance before, during, and after any drill-through operations.

The field injection port 200 and its orientation further defines the orientation of the predetermined path 202. The predetermined path 202 defines the drilling implement orientation, such that a result of a drilling operations is a precisely targeted injection bore 203 or via that directs an injectable repair fluid composition from an external injection fitting, through the field injection port 200, and into the targeted location 204, such as the packing chamber 124, the gasket interface 140, or the valve seat 150. See, FIGS. 4-13, for examples. That is to say that the field injection port 200 comprises an orientation that guides a drilling implement placed therein along the predetermined path 202 that intersects the targeted location 204 within the valve assembly 100. The targeted location 204 may be disposed at or adjacent to the packing chamber 124, at or adjacent to the valve seat 150, or at or adjacent to a gasket interface 140, depending on the embodiment. In some embodiments, the field injection port 200 accommodates a depth-controlled drilling implement configured to limit drilling penetration to the targeted location 204. In some embodiments, this is effectuated with a depth stop, such as a collar situated at a predetermined distance from the tip of the drilling implement, for example. In such cases, the collar would contact a limiting surface, such as the body 102, and thus be physically prevented from drilling any deeper therein. In other embodiments, the depth may be controlled by markings or features on a drilling implement. In other embodiments, the apparatus holding the drilling implement serves as the depth stop itself. In some embodiments, the field injection port 200 is positioned such that the predetermined path 202 is generally normal to a local outer surface of the body 102. In some embodiments, the predetermined path 202 is oriented at a non-normal angle chosen to intersect the targeted location 204 at a desired point.

In some embodiments, the body 102 comprises the valve body 104, and the field injection port 200 is formed in the valve body 104. In some embodiments, the body 102 comprises a region proximate a flange 154, and the field injection port 200 is formed in the region proximate the flange 154. In some embodiments, the body 102 comprises the bonnet 106, and the field injection port 200 is formed in the bonnet 106. In some embodiments, the valve assembly has a plurality of field injection ports 200. In some embodiments, the plurality of field injection ports 200 are directed to the same packing, gasket, or targeted feature of the valve assembly 100, with the packing, gasket, valve seat, or other targeted feature each having a plurality of associated targeted locations. For example, a valve assembly 100 may have opposing field injection ports 200 that are each directed to a different targeted location of the same packing 126. This may also be the case for a gasket 142, valve seat 150, or other targeted features. In some embodiments, a plurality of field injection ports 200 are directed to different packing 126, gasket 142, valve seat 150, or other targeted features of the valve assembly 100. For example, a valve assembly 100 may have one or more field injection ports 200 targeting a packing 126, and/or one or more field injection ports 200 targeting a gasket 142, and/or one or more field injection ports 200 targeting a valve seat 150, and/or one or more field injection ports 200 targeting another feature.

In some embodiments, the targeted location 204 comprises the packing 126 disposed in the packing chamber 124. In some embodiments, the targeted location 204 comprises the valve seat 150 associated with the valve member 110. In some embodiments, the targeted location 204 comprises one or more gaskets 142. Gaskets may be at junctions between the valve assembly 100 and other members, such as pipeline flanges. Gaskets may be at junctions between portions the valve assembly 100. In particular, the gaskets 142 may include the body-to-bonnet gasket 144 disposed between the valve body 104 and the bonnet 106, the body-to-flange-end gasket 146 disposed between a flange of the valve body 104 and a mating pipeline flange, the pressure seal gasket 148 disposed in a pressure seal bonnet configuration, and other gaskets associated with valve assemblies 100.

The field injection port 200 allows injection of repair fluid compositions into the packing chamber 124 or other targeted locations 204 of the valve assembly 100 during operation when the packing 126, valve seat 150, or gaskets 142, 144, 146, 148 are leaking or degraded. By providing the field injection port 200 with a factory-defined orientation, internal threads 206, chip chamber 208, and predetermined wall thickness, the valve assembly 100 improves safety during field drilling and injection procedures, reduces reliance on ad hoc drill-and-tap operations, avoids hole-starting on curved or irregular surfaces, and increases the likelihood of successful leak mitigation while maintaining pressure boundaries and code compliance.

This field injection port 200 allows the precisely targeted injection of a repair fluid in-place, without taking a valve out of service, such that leaks of packing, seals, and gaskets are more easily, faster, less expensive, more accurately, and safer than prior art solutions. External valve leakage to atmosphere and/or internal bypass leakage is reduced, thereby mitigating fugitive emissions and loss of process fluid. By enabling controlled in-situ injection during operation, the design prolongs valve service life, enhances personnel and process safety during hot drill-and-tap or field injection procedures, and supports compliance with applicable valve and pressure-boundary codes.

### Methods of Manufacture

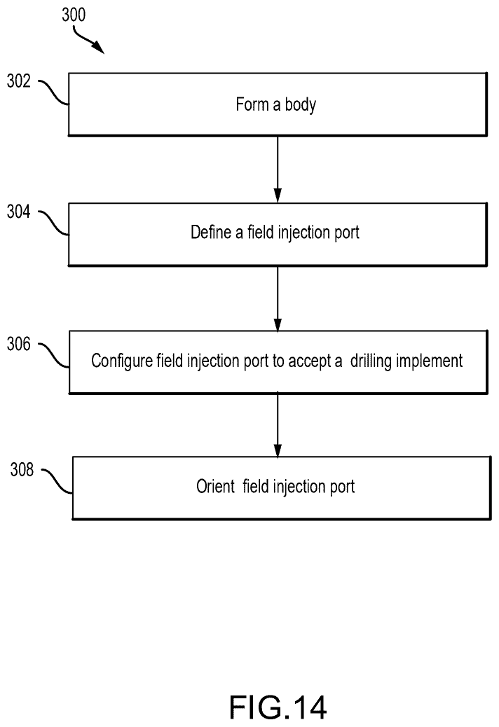

With continued reference to FIGS. 1-13 in addition to FIG. 14, embodiments also relate to a method of manufacturing a valve assembly 100 configured for field injection repair of internal sealing components. FIG. 14 is a flow chart illustrating a method 300 of manufacturing a valve assembly 100 according to an embodiment. The method may be carried out in conjunction with the manufacture of new valve assemblies 100 or during remanufacture or upgrade of existing valve assemblies that are brought into compliance with applicable pressure boundary standards.

In some embodiments, as illustrated by step 302, the method includes forming the body 102. This may be as a monolithic or multi-piece structure configured to define the fluid passage 108, stem passage 122, and other internal cavities and/or features. The body 102 may include the valve body 104, the bonnet 106, and one or more flanges 154, which may be integrally formed or attached by welding, bolting, threading, or other joining techniques. After forming the body 102, the method may further comprises assembling the valve assembly 100 using the body 102, in some embodiments. This may include, among other steps and components, installing the valve member 110, stem member 120, packing 126, gasket(s) 142, associated hardware such as the gland assembly 128 and mechanical fasteners 112, etc.

In some embodiments, the body 102 is formed using one or more conventional metalworking processes including casting, forging, drilling, threading, and machining. Othe manufacturing processes are contemplated. For example, the valve body 104 and bonnet 106 may be cast or forged to near-net shape, followed by machining of the fluid passage 108, stem passage 122, gasket seating surfaces, and/or flange faces to achieve the required dimensional tolerances, surface finishes, requisite structural integrity, etc. In some embodiments, the body 102 is at least partially formed using an additive manufacturing process, such as powder bed fusion, directed energy deposition, or binder jetting, for example, and without limitation. Additive manufacturing may facilitate the formation of internal features including complex chip chambers 208, contoured drilling paths 202, and integral guide chambers 216 that would be difficult or impractical to create using only subtractive operations.

In some embodiments, and illustrated by step 304, the field injection port 200 is defined in the body 102. The manufacturing processes by which the field injection port 200 is defined are exemplified herein. In some embodiments, at least one field injection port 200 in the bonnet 106. For example, the field injection port 200 may be located in a bonnet wall region. This may be proximate the packing chamber 124 so that the predetermined path 202 passes through the wall 152 and intersects the packing 126 at the targeted location 204. In some embodiments, the body 102 comprises the valve body 104, and the method includes defining the field injection port 200 in the valve body 104, such as in a wall segment adjacent to a valve seat 150 or near a body-to-flange-end gasket 146 as illustrated in FIGS. 9-10 and 13. In some embodiments, the body 102 comprises a flange 154, and the method includes defining the field injection port 200 in the flange region so that the predetermined path 202 intersects a gasket interface 140 between a valve flange and a mating pipeline flange.

The process of defining the field injection port 200 may occur during primary body manufacture or in a secondary operation. For example, in some embodiments a rough port cavity is formed during casting or forging, and subsequent machining operations size and finish the field injection port 200 to establish the aperture 210, chip chamber 208 if any, guide chamber 216, and terminus 212. In some embodiments, the field injection port 200 is entirely created as a machined feature in a solid or pre-formed body 102, using drilling, boring, reaming, and threading operations to achieve the desired geometry and dimensional tolerances. Additive manufacturing may also be employed for all embodiments.

In some embodiments, the field injection port 200 is configured so that it may accept a drilling implement, such as in step 306. In embodiments, this may include sizing the aperture 210 and internal bore geometry to receive a standard drilling tool, such as a twist drill bit or pilot drill for example and without limitation, as well as any associated drilling guide or injection fitting that may be temporarily or permanently installed in the field injection port 200. The field injection port 200 may be oriented such that its longitudinal axis defines the predetermined path 202. The field injection port 200 may include an internal guide chamber 216 that has a bore aligned with the predetermined path 202, with the bore configured to accept the drilling implement.

Turning to step 308, the orientation of the field injection port 200 is predetermined during design and manufacture so that, when a drilling implement is inserted through the field injection port 200 and advanced into the wall 152, the drilling implement follows the predetermined path 202 and intersects the targeted location 204 at a defined entry region. In some embodiments, the predetermined path 202 is generally normal to the local external surface of the body 102 at the aperture 210, providing a stable seating surface for drilling fixtures. In some embodiments, the predetermined path 202 is intentionally oriented at a non-normal angle to avoid internal structures, to intersect a seat pocket, or to reach a specific region of a packing chamber 124 or gasket interface 140.

In some embodiments, the targeted location 204 comprises a packing 126 disposed in the annular packing chamber 124 between the stem member 120 and the stem passage 122. In such cases, the method includes locating and orienting the field injection port 200 so that the predetermined path 202 passes through the wall 152 and terminates within the packing chamber 124 at a region where injectable repair fluid compositions can effectively distribute around the stem member 120. In some embodiments, the targeted location 204 comprises the valve seat 150 associated with the valve member 110; here the field injection port 200 is positioned and oriented such that the predetermined path 202 intersects a seat pocket or seat gasket region for delivery of sealant or packing material.

In some embodiments, the targeted location 204 comprises one or more gaskets 142. For example, the method may involve orienting the field injection port 200 so that the predetermined path 202 intersects a body-to-bonnet gasket 144 disposed between the valve body 104 and the bonnet 106, a body-to-flange-end gasket 146 disposed between a flange on the valve body 104 and a mating pipeline flange, or a pressure seal gasket 148 used in a pressure-seal bonnet configuration. The field injection port 200 can be designed to intersect a single gasket 142 or multiple gaskets 142 by using multiple field injection ports 200 directed at different targeted locations 204 or by employing branched or contoured internal drilling paths created via additive manufacturing.

In some embodiments, defining the field injection port 200 includes defining a chip chamber 208 between the aperture 210 and the terminus 212 of the field injection port 200. The chip chamber 208 is formed with a cross-sectional area greater than that of a nominal drilling implement to provide chip clearance during field drilling operations and to accommodate the accumulation of chips and swarf generated as the drilling implement penetrates the wall 152. The chip chamber 208 may be formed by undercutting or otherwise enlarging a portion of the field injection port 200 deep to the guide chamber 216 such that the drilling implement remains guided while debris is allowed to migrate into the chip chamber 208.

In some embodiments, the method further includes forming a guide chamber 216 in fluid isolation from the pressure boundary interior until drill-through is performed, the guide chamber 216 being configured to closely receive the drilling implement and constrain lateral motion so that the drilled injection bore 203 follows the predetermined path 202. The guide chamber 216 may be integral with or downstream of the chip chamber 208. In embodiments that include both a chip chamber 208 and a guide chamber 216, the chip chamber 208 provides debris storage while the guide chamber 216 provides alignment and directional control.

In some embodiments, defining the field injection port 200 further comprises providing a wall 152 defined by the body 102 between the terminus 212 of the field injection port 200 and the targeted location 204. The wall 152 is generally a material span having a predetermined wall thickness between the terminus 212 and the targeted location 204. During design and manufacture, the predetermined wall thickness is selected to meet or exceed minimum wall thickness requirements for pressure-retaining components established by one or more applicable standards as discussed herein.

In some embodiments, the method includes calculating the minimum wall thickness using design rules that account for valve size, pressure class, material group, and the diameter or geometry of the pressure boundary region into which the field injection port 200 is formed. The method may include performing analytical or numerical stress evaluation, such as finite element analysis for example, related to the region surrounding the field injection port 200, the chip chamber 208, and the wall 152, etc. to confirm that the addition of the field injection port 200 does not compromise pressure boundary integrity under specified design and test conditions. The location, size, depth, and orientation of the field injection port 200 may be iteratively adjusted during design to maintain or exceed the required wall thickness and stress limits after accounting for material removal associated with both the field injection port 200 and the subsequently drilled injection bore 203.

In some embodiments, the method includes threading the field injection port 200 to define internal threads 206 configured to engage mating components such as injection fittings, temporary drilling guides, and plugs 214. The threading operation may be performed using taps, thread milling, or other threading processes compatible with the body material and geometry. The internal threads 206 may be National Pipe Taper (NPT) threads or other threaded configurations designed for high-pressure service, allowing installation of standardized fittings and accessories.

In further embodiments, the method includes removably installing a plug 214 into the threaded field injection port 200 after the internal threads 206 are formed. The plug 214 may be a threaded metal plug or other closure member with external threads complimentary to the internal threads 206, and may incorporate sealing features such as metal-to-metal seats, thread sealants, gaskets, etc. Installing the plug 214 during manufacture protects the internal threads 206 from damage caused by handling, coating, or debris ingress of the field injection port 200 until such time as field drilling or injection operations are required. After completion of field operations, the plug 214 may be reinstalled or replaced to re-establish a sealed closure.

Although drilling is typically carried out in the field, the method of manufacturing may also include providing features configured to cooperate with depth-controlled drilling implements and injection fittings during later service. For example, the orientation, length, and diameter of the field injection port 200 may be selected to accommodate a drilling tool having a depth stop collar or shoulder that contacts a limiting surface at or near the aperture 210 when the drilling implement has advanced through the wall 152 to the targeted location 204. This arrangement provides an inherent physical depth limit determined during manufacture, thereby reducing the risk of over-penetration or damage to internal components when the drilled injection bore 203 is formed.

In some embodiments, the manufacturing method further includes marking or otherwise indicating on the external surface of the body 102 the intended drilled injection bore 203 depth, tool size, or other field-use parameters associated with the field injection port 200. Such markings may include engraved depth values, code symbols, or identification of the targeted location 204 (for example, “PACKING,” “SEAT,” or “GASKET”) to provide guidance to field technicians and to further reduce the likelihood of procedural errors during drill-and-inject operations.

### Methods of Repair

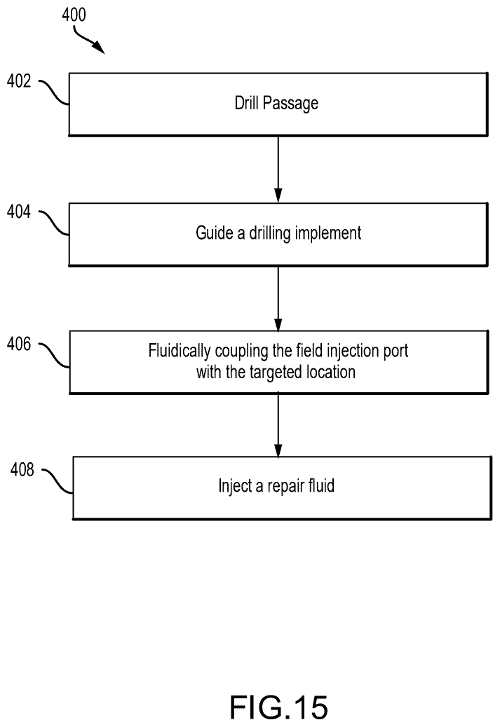

With continued reference to FIGS. 1-14, in addition to FIG. 15, embodiments also relate to a method of repairing a valve assembly 100 having the body 102 with the field injection port 200 defined therein during manufacture. FIG. 15 is a flow chart illustrating the method of repairing a valve assembly 100. In step 402, a injection bore 203 is drilled in the body through a field injection port 200. A drilling implement, such as a twist drill bit or other suitable drilling tool, drills into the body. This may occur while the valve assembly 100 is installed in a process line. Suitable drilling implements are as described herein. The field injection port 200 guides the drilling implement along the predetermined path 202 in the body 102 to form the drilled injection bore 203 that intersects the targeted location 204 in step 404. Connecting the terminus 212 of the field injection port 200 to the targeted location 204 is effectuated by the drilled injection bore 203 in step 406. Following formation of the drilled injection bore 203, the method further comprises injecting a repair fluid into the field injection port 200 so that the repair fluid flows through the drilled injection bore 203 and into the targeted location 204, as illustrated in step 408. The repair fluid distributes around the packing 126, valve seat 150, and/or gaskets 142, 144, 146, 148 or other internal sealing features, depending on the embodiment, thereby preventing, mitigating, and/or arresting leakage while maintaining the pressure boundary of the valve assembly 100.

In some embodiments, the body 102 comprises the valve body 104, and the field injection port 200 is formed in a wall region of the valve body 104 adjacent to the valve seat 150 or a body-to-flange-end gasket 146 so that the predetermined path 202 intersects the associated targeted location 204. In other embodiments, the body 102 comprises a flange 154 and the field injection port 200 is provided in the flange region, such that the drilled injection bore 203 intersects a gasket interface 140 between the flange 154 and a mating pipeline flange. While, in some embodiments, the body 102 comprises the bonnet 106 and the field injection port 200 is located in a bonnet wall region so that the predetermined path 202 intersects the packing chamber 124 and the packing 126 disposed therein. The field injection port 200 may thus be selectively located in the valve body 104, the flange 154, the bonnet 106, or combinations thereof, depending on whether the intended targeted location 204 is a packing 126, the valve seat 150, or one or more gaskets 142 of the valve assembly 100. Of course, in some embodiments, more than one field injection port 200 is contemplated in a single body 102.

In some embodiments, a method further includes collecting chips produced by drilling into the body 102. The chips are collected in the chip chamber 208 defined by the field injection port 200 between the aperture 210 and the terminus 212. During drilling, the drilling implement advances from the guide chamber 216 into the wall 152 and generates metal chips and swarf, which migrate into and accumulate within the chip chamber 208 whose increased cross-sectional area provides chip clearance and reduces the likelihood of drill jamming while maintaining a sealed environment. In some embodiments, the chip chamber 208 is sized such that the expected chip volume corresponding to the drilled injection bore 203 geometry is contained therein, thereby minimizing the risk that chips will enter the packing chamber 124, the valve seat 150 region, or the gasket interface 140, depending on the embodiment, thus reducing potential interference with sealing performance.

In some embodiments, the method includes removing a plug 214 that was preinstalled in the field injection port 200 prior to performing the drilling and/or injection operations. The plug 214, which may be threadingly engaged with the internal threads 206 of the field injection port 200, is unscrewed or otherwise disengaged to open access to the guide chamber 216, after which a drilling guide, depth-controlled drilling implement, or injection fitting may be installed in the field injection port 200 for subsequent drilling and repair fluid injection steps. After completion of the injection operation, the method may further include reinstalling the plug 214 or installing a replacement closure member into the threaded field injection port 200 to restore a sealed condition at the aperture 210.

In some embodiments, the targeted location 204 comprises at least one of the packing 126, the valve seat 150, and the gasket(s) 142 of the valve assembly 100. For example, in an embodiment the predetermined path 202 is oriented such that the drilled injection bore 203 terminates in the packing chamber 124 at a region where the repair fluid can encircle the stem member 120 and permeate the packing 126. In some embodiments, the predetermined path 202 intersects the valve seat 150 or a seat pocket region to deliver repair fluid that fills leakage pathways at the seating interface between the valve member 110 and the valve seat 150. In further embodiments, the targeted location 204 comprises one or more gaskets 142, including a body-to-bonnet gasket 144 between the valve body 104 and the bonnet 106, a body-to-flange-end gasket 146 between a flange of the valve body 104 and a mating pipeline flange, or a pressure seal gasket 148 used in a pressure-seal bonnet configuration, and the field injection port 200 is positioned so that the drilled injection bore 203 intersects the gasket interface 140 at or near the gasket 142 to allow introduction of repair fluid around the gasket perimeter. Other locations and seal types are also considered.

In some embodiments, the drilled injection bore 203 passes through the wall 152 defined by the body 102. The wall 152 is the area between the terminus 212 of the field injection port 200 and the targeted location 204, wherein the wall 152 comprises the material span corresponding to the predetermined wall thickness between the terminus 212 and the targeted location 204, as described herein. The predetermined wall thickness is selected during design and manufacture such that the material span of the wall 152 remaining after formation of the field injection port 200 meets or exceeds a minimum wall thickness that satisfies applicable code requirements for pressure-retaining components, including standards provided by at least one of ASME (for example ASME B16.34), API (for example API 600, 603, 594, 6D), ISO (for example ISO 17292, 10434), and MSS. In some embodiments, analytical calculations and/or numerical stress evaluations such as finite element analysis are employed to verify that the local stresses in the region surrounding the field injection port 200, chip chamber 208, wall 152, and drilled injection bore 203 remain within allowable limits under design and test pressures during and after the repair operation, thereby ensuring that the repair method preserves pressure boundary integrity and maintains valve code compliance.

It will be understood that the terms such as “approximately,” “about,” and “substantially” as used within the specification and the claims herein allows for a certain amount of variation from any exact dimensions, measurements, and arrangements, and that those terms should be understood within the context of the description and operation of the embodiments as disclosed herein.

It will further be understood that terms such as “top,” “topside,” “bottom,” “above,” and “below” and similar terms as used within the specification and the claims herein are terms of convenience that denote the spatial relationships of parts relative to each other rather than to any specific spatial or gravitational orientation. Thus, the terms are intended to encompass an assembly of component parts regardless of whether the assembly is oriented in the particular orientation shown in the drawings and described in the specification, upside down from that orientation, or any other rotational variation.

It will also be understood that terms such as “flat” as used within the specification and the claims herein are terms that convey a general shape. These are approximations towards being flat and/or planar, but encompass variations including materials with textures, perforations, holes, etchings, manufacturing variations, and relatively minor curvatures or other non-flat and/or non-planar features.

It will be appreciated that the term “embodiment” as used herein should not be construed to mean that only a single embodiment having a single essential element or group of elements is presented. Similarly, it will also be appreciated that the term “present embodiment(s)” encompasses a number of separate innovations which can each be considered a separate embodiment. Although the present embodiments have been described in detail with regard to the embodiments and drawings thereof, it should be apparent to those skilled in the art that various adaptations and modifications of the present embodiments may be accomplished without departing from the spirit and the scope of the embodiments. Indeed, persons skilled in the art will recognize that certain elements of the above-described embodiments may variously be combined or eliminated to create further embodiments, and such further embodiments fall within the scope and teachings of the present description. It will also be apparent to those of ordinary skill in the art that the above-described embodiments may be combined in whole or in part to create some embodiments within the scope and teachings of the present description.

Accordingly, it is to be understood that the detailed description and the accompanying drawings as set forth hereinabove are not intended to limit the breadth of the present embodiments. Although specific embodiments are described herein for illustrative purposes, various equivalent modifications are possible within the scope of the present description, as those skilled in the relevant art will recognize. The teachings provided herein can be applied to other coil applications, and not just to the embodiments described above and shown in the accompanying figures.

We claim:

1. A method of manufacturing a valve assembly, comprising:
forming a body;
defining a field injection port in the body;
configuring the field injection port to accept a drilling implement;
orienting the field injection port to guide the drilling implement along a predetermined path that intersects a targeted location in the valve assembly.

2. The method of claim 1, further comprising assembling the valve assembly using the body.

3. The method of claim 1, wherein the body comprises a bonnet, and further comprising defining the field injection port in the bonnet.

4. The method of claim 1, wherein the body comprises a valve body, and further comprising defining the field injection port in the valve body.

5. The method of claim 1, wherein the body comprises a flange in body, and further comprising defining the field injection port in the flange.

6. The method of claim 1, wherein the targeted location comprises a packing.

7. The method of claim 1, wherein the targeted location comprises a valve seat.

8. The method of claim 1, wherein the targeted location comprises a gasket.

9. The method of claim 8, wherein the gasket comprises a body bonnet gasket.

10. The method of claim 8, wherein the gasket comprises a body flange end gasket.

11. The method of claim 8, wherein the gasket comprises a pressure seal gasket.

12. The method of claim 1, wherein defining the field injection port in the body further comprises at least partially defining the field injection port using at least one of casting, forging, drilling, threading, and machining.

13. The method of claim 1, wherein defining the field injection port in the body further comprises defining a guide chamber configured to further guide the drilling implement.

14. The method of claim 1, comprising defining the field injection port in the body by at least partially forming the body with an additive manufacturing process.

15. The method of claim 1, comprising defining a chip chamber between an aperture of the field injection port and a terminus of the field injection port.

16. The method of claim 1, further comprising providing a wall defined by the body, the wall disposed between a terminus of the field injection port and the targeted location, wherein the wall comprises a material span corresponding to a predetermined wall thickness between the terminus and the targeted location.

17. The method of claim 16, wherein the material span comprises a minimum wall thickness that meets or exceeds standards provided by at least one of ASME, API, ISO, and MSS.

18. The method of claim 1, comprising threading the field injection port to define threads therein.

19. The method of claim 18, comprising:
removably installing a plug into the field injection port; and
protecting the threads from damage with the plug.

20. A valve assembly (100), comprising:
a body (102); and
a field injection port (200) formed with the body (102) during manufacturing of the body (102), wherein the field injection port (200) comprises an orientation that guides a drilling implement placed therein along a predetermined path (202) to intersect a targeted location (204).

21. The valve assembly (100) of claim 20, wherein the body (102) comprises a valve body (104) further comprising the field injection port.

22. The valve assembly (100) of claim 20, comprising a guide chamber of the field injection port (200) configured to further guide the drilling implement.

23. The valve assembly (100) of claim 20, wherein the body (102) comprises a body (102) flange that further comprises the field injection port.

24. The valve assembly (100) of claim 20, wherein the body (102) comprises a bonnet (106) that further comprises the field injection port.

25. The valve assembly (100) of claim 20, wherein the targeted location (204) comprises a packing.

26. The valve assembly (100) of claim 20, wherein the targeted location (204) comprises a valve seat.

27. The valve assembly (100) of claim 20, wherein the targeted location (204) comprises a gasket.

28. The valve assembly (100) of claim 27, wherein the gasket comprises a body (102) bonnet (106) gasket.

29. The valve assembly (100) of claim 27, wherein the gasket comprises a body (102) flange end gasket.

30. The valve assembly (100) of claim 27, wherein the gasket comprises a pressure seal gasket.

31. The valve assembly (100) of claim 20, wherein the field injection port (200) is at least partially formed in the body (102) using at least one of casting, forging, drilling, threading, and machining.

32. The valve assembly (100) of claim 20, wherein the field injection port (200) is at least partially formed in the body (102) with an additive manufacturing process.

33. The valve assembly (100) of claim 20, wherein a chip chamber (208) is defined between an aperture of the field injection port and a terminus of the field injection port (200), wherein the chip chamber (208) comprises a space having a cross-sectional area greater than a cross-sectional area of the drilling implement, wherein the chip chamber (208) is configured to capture chips created during a drilling operation.

34. The valve assembly (100) of claim 20, comprising threads defined in field injection port (200).

35. The valve assembly (100) of claim 20, wherein the valve assembly (100) comprises at least one of a gate valve, a globe valve, a check valve, a ball valve, a butterfly valve, and a plug valve.

36. The valve assembly (100) of claim 20, wherein a wall defined by the body (102) is disposed between a terminus of the field injection (200) port and the targeted location, wherein the wall comprises a material span corresponding to a predetermined wall thickness between the terminus and the targeted location (204).

37. The valve assembly (100) of claim 36, wherein the material span comprises a minimum wall thickness that meets or exceeds standards provided by at least one of ASME, API, ISO, and MSS.

38. A method of repairing a valve assembly having a body, comprising:
drilling an injection bore in the body through a field injection port with a drilling implement, the field injection port being defined by the body during manufacturing of the valve assembly; and
guiding the drilling implement, with the field injection port, along a predetermined path in the body to form a drilled injection bore that intersects a targeted location;
fluidically coupling the field injection port with the targeted location with the drilled injection bore; and
injecting a repair fluid into the field injection port and subsequently into the targeted location via the drilled injection bore.

39. The method of claim 38, wherein the body comprises a valve body that further comprises the field injection port.

40. The method of claim 38, wherein the body comprises a body flange that further comprises the field injection port.

41. The method of claim 38, wherein the body comprises a bonnet that further comprises the field injection port.

42. The method of claim 38, further comprising collecting chips produced by drilling into in the body, wherein the chips are collected in a chip chamber defined by the field injection port.

43. The method of claim 38, wherein guiding the drilling implement, with the field injection port, further comprises guiding the drilling implement with a guide chamber of the field injection port.

44. The method of claim 38, further comprising removing a plug preinstalled in the field injection port.

45. The method of claim 38, wherein the targeted location comprises a packing.

46. The method of claim 38, wherein the targeted location comprises a valve seat.

47. The method of claim 38, wherein the targeted location comprises a gasket.

48. The method of claim 47, wherein the gasket comprises a body bonnet gasket.

49. The method of claim 47, wherein the gasket comprises a body flange end gasket.

50. The method of claim 47, wherein the gasket comprises a pressure seal gasket.

51. The method of claim 38, wherein the targeted location comprises at least one of a packing and a gasket of the valve assembly.

52. The method of claim 38, wherein the drilled injection bore passes through a wall defined by the body, wherein the wall comprises a material span corresponding to a predetermined wall thickness between the terminus and the targeted location.

53. The method of claim 52, wherein the material span comprises at least a minimum wall thickness that meets or exceeds standards provided by at least one of ASME, API, ISO, and MSS.
